(19) United States
(12) Patent Application Publication
(10) Pub. No.: US 2024/0255330 A1
(43) Pub. Date: Aug. 1, 2024

(54) METHOD AND DEVICE FOR ANALYSING A DEVICE FOR SPRAYING A PHARMACEUTICAL FLUID PRODUCT

(71) Applicant: APTAR FRANCE SAS, LE NEUBOURG (FR)

(72) Inventor: Remy WYART, BALINES (FR)

(73) Assignee: APTAR FRANCE SAS, LE NEUBOURG (FR)

(21) Appl. No.: 18/290,054

(22) PCT Filed: Feb. 28, 2022
(86) PCT No.: PCT/FR2022/050354

§ 371 Date: Nov. 9, 2023

(30) Foreign Application Priority Data
May 10, 2021 (FR) FR2104936

(51) Int. Cl. G01F 1/68; B05B 12/18; G01F 1/76

(52) U.S. Cl. CPC G01F 1/68; B05B 12/18; G01F 1/76

(57) ABSTRACT

A method for analysing a fluid product spray device having the following steps: providing a spray head (1) for a device for spraying a pharmaceutical fluid product, the spray head (1) having a spray orifice (2); providing a receiving surface (10) having a plurality of sensors (20); passing a flow of compressed gas (F) through the spray orifice (2) of the spray head (1); sending the flow of compressed gas (F) onto the receiving surface (10); visualising the impact zone for the flow of compressed gas (F) on the receiving surface (10) by the sensors (20); and analysing the visualisation of the impact zone in order to determine whether or not the impact zone complies with predetermined specifications. The receiving surface (10) is at least partially spherical in shape.

The present invention relates to a device and to a method for analysing a spray generated by a device for spraying a pharmaceutical fluid product.

Spray devices for spraying pharmaceutical fluid product are well known. They generally comprise a spray head provided with a spray orifice, assembled on a reservoir containing the fluid product to be distributed. Particularly in nasal spray applications, the therapeutic effectiveness of the sprayed fluid product may depend on the properties of the spray generated while the device is being actuated. At the end of the assembly line, i.e. once the spray device has been assembled and just prior to being sent to the pharmaceutical fluid product manufacturer for assembly there onto a corresponding reservoir, it is known for a certain number of samples of assembled devices to be laboratory tested in order to check whether the properties of the spray correspond to pre-defined production specifications.

A disadvantage with that system is that it pertains to assembled devices, and thus destroys those devices which, after having been tested, can no longer be delivered to the customer.

Furthermore, the system requires human verification of the tested devices, and is thus not suitable for being completely automated.

To overcome this drawback, the document WO 2018/130791 proposes visualization of a flow of hot or cold compressed air sent through a spray head by strioscopy. This method makes it possible to distinguish a spray from a jet, but does not make it possible to evaluate the geometry and/or symmetry of the spray, and therefore its compliance with given specifications. This method also has the disadvantage of having to provide a strioscopic bench, which is relatively complex and expensive.

Documents U.S. 2016/216108, JPS 54 127 347 et U.S. Pat. No. 5,753,806 describe other prior-art devices.

An object of the present invention is to overcome the above-mentioned drawbacks.

In particular, an object of the present invention is to provide a device and a method for analysing a device for spraying a pharmaceutical fluid product that makes it possible to detect the devices that are non-compliant with predetermined specifications.

An object of the present invention is also to provide a device and a method for analysis that do not destroy the tested devices.

An object of the present invention is also to provide a device and a method for analysis which is substantially automated.

An object of the present invention is also to provide a device and method for analysis which makes it possible to test a large number, in particular 100%, of the spraying devices, without slowing down the assembly line to a substantial extent.

An object of the present invention is also to provide a device and method for analysis which is simple and/or inexpensive to manufacture, assemble and use.

Thus, the present invention relates to a method for analysing a device for spraying a pharmaceutical fluid product, comprising the following steps:
providing a spray head for a device for spraying a pharmaceutical fluid product, said spray head comprising a spray orifice;
providing a receiving surface comprising a plurality of sensors,
passing a flow of compressed gas through said spray orifice of said spray head,
sending said flow of compressed gas onto said receiving surface,
visualising the impact zone for said flow of compressed gas on said receiving surface by means of said sensors, and
analysing said visualisation of said impact zone in order to determine whether or not said impact zone complies with predetermined specifications,
said receiving surface being at least partially spherical in shape.

Advantageously, said flow of compressed gas is a flow of compressed air.

Advantageously, said step for analysis comprises determining the geometry, in particular the symmetry, of the impact zone for said flow of compressed gas on said receiving surface.

Advantageously, said predetermined specifications comprise a predetermined planar extent of the impact zone for said flow of compressed gas on said receiving surface, in a manner such that the spray heads for which said planar extent is similar to said predetermined planar extent are classified as compliant, and the spray heads for which said planar extent is different from said predetermined planar extent are classified as non-compliant.

Advantageously, said receiving surface is formed by the inner surface of a part of a sphere, in particular a half-sphere.

Advantageously, said receiving surface comprises a plurality of openings, each being connected to a respective sensor.

Advantageously, said sensors form a honeycomb array comprising at least two concentric sensor zones.

The present invention also relates to a device for analysing a device for spraying a pharmaceutical fluid product, comprising:
a spray head for a device for spraying a pharmaceutical fluid product, said spray head comprising a spray orifice;
a receiving surface comprising a plurality of sensors,
means for generating a flow of compressed gas in order to pass a flow of compressed gas through said spray orifice of said spray head onto said receiving surface,
visualisation means in order to visualize through said sensors the impact zone for said flow of compressed gas on said receiving surface, and
means for analysis for the analysis of said visualisation of said impact zone in order to determine whether or not said impact zone complies with predetermined specifications, said receiving surface being at least partially spherical in shape.

Advantageously, said flow of compressed gas is a flow of compressed air.

Advantageously, said receiving surface is formed by the inner surface of a part of a sphere, in particular a half-sphere.

Advantageously, said receiving surface comprises a plurality of openings, each being connected to a respective sensor.

Advantageously, a filter is arranged in front of each sensor.

Advantageously, said sensors form a honeycomb array comprising at least two concentric sensor zones.

Advantageously, said sensor array comprises a central area with a single central sensor, and a first concentric area, arranged radially outside of said central area, with six sensors.

Advantageously, said sensor array comprises a second concentric zone, arranged radially outside of said first concentric zone, with twelve sensors.

Advantageously, said sensor array comprises a third concentric zone, arranged radially outside of said second concentric zone, with eighteen sensors.

Advantageously, said sensors are close to a another, thus forming a regular and dense array of sensors zones on said receiving surface.

Advantageously, said means for generating a flow of compressed gas are adapted to generate pulses of adjustable duration, in particular from 50 to 300 ms.

Advantageously, said sensors are mass flow sensors.

These and other characteristics and advantages will appear more clearly from the following detailed description made with reference to the accompanying drawings given by way of non-limiting examples, and in which:

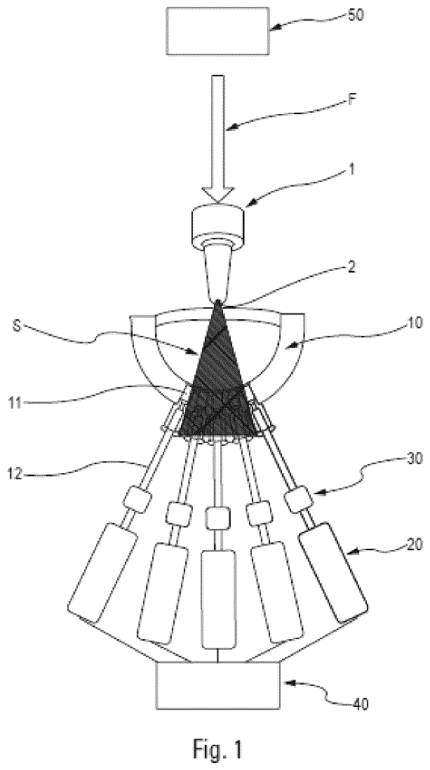

FIG. 1 is a schematic view of a device for analysing a spraying device, according to an advantageous embodiment,

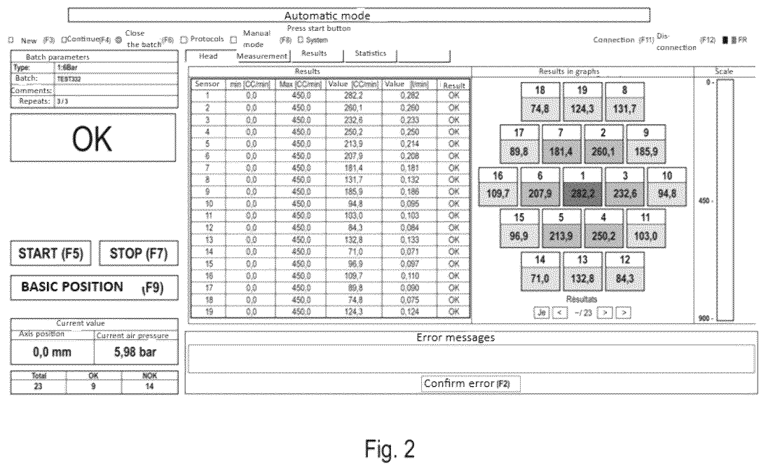

FIG. 2 is a schematic representation of a compliant impact zone.

One object of the invention is to improve the quality of spray device inspection.

In conventional manner, each spray device comprises a spray head 1 provided with a spraying orifice 2. In general, a spray profile (not shown) may be provided upstream of said spray orifice 2 in order to generate a spray at the outlet from the orifice.

The present invention envisages passing a flow of compressed gas F through each spray head 1, and directing this flow F leaving the spray orifice 2 in the form of a conical spray S, towards a receiving surface 10, comprising a plurality of sensors 20. Advantageously, the flow of compressed gas F is a flow of compressed air, but it should be understood that, in accordance with the invention, any suitable gas other than air could be used.

FIG. 1 shows a test device according to an advantageous embodiment.

The spray head 1 is disposed opposite a receiving surface 10 of at least partially spherical shape. In particular, the receiving surface 10 may be formed by the inner surface of a part of a sphere, for example a half-sphere, as can be seen in FIG. 1.

In the example in FIG. 1, the receiving surface 10 comprises a plurality of openings 11, each being connected to a respective sensor 20, for example via a respective pipe 12. Alternatively, the sensors 20 could be on the receiving surface 10. Because of the hemispherical shape of the receiving surface 10, each opening 11 is disposed at the same distance from the spray orifice 2.

Optionally, a filter 30 may be arranged in front of each sensor 20.

Means 50 for generating a flow of compressed gas F are provided in order to cause a flow of compressed gas F to pass through the spray head 1.

Advantageously, the sensors and/or openings form a honeycomb array comprising at least two concentric sensor zones. Thus, the sensor array 20 may include a central area with a single central sensor, and a first concentric area, arranged radially outside of said central area, with six sensors.

Advantageously, the sensor array 20 comprises a second concentric zone, arranged radially outside of said first concentric zone, with twelve sensors. It is this configuration with nineteen sensors that is used for the analysis shown in FIG. 2.

Optionally, the sensor array 20 may include a third concentric zone, arranged radially outside of said second concentric zone, with eighteen sensors.

Other forms of array, e.g. in the form of a square or circle, can also be envisaged.

The particular shape of the receiving surface 10, with a plurality of sensors 30 close to one another, thus forming a regular and dense array on said receiving surface, means that a local contact can be made by the flow of compressed gas F on the sensors 20, without dispersions and without perturbations to the flow, which makes the impact zone visible with great reliability.

Advantageously, the sensors 20 are mass flow sensors. The choice of a mass flow control, based on the principle of mass conservation, is the most judicious. Specifically, since gas, and in particular air, is a compressible fluid, it would not be relevant to control a volume flow rate. A mass flow sensor uses the calories of the fluid (gas or liquid) as conductivity to determine the mass flow. The sensor is composed of a tube in which two stainless steel temperature probes and a heating element are located. The one probe measures the temperature of the gas before the heating element and the other measures the temperature of the gas after the heating element. A temperature difference ΔT is thus created between the two probes. Without a flow, this ΔT does not change. When a flow exists, the heating element returns a quantity of heat to the flowing gas. This heat loss, identified through the probes, is compensated by providing more energy to the heating element in order to maintain a constant ΔT between the two probes. The energy required to maintain this ΔT is proportional to the mass flow. By measuring the energy consumed by the probe it is then possible to determine the mass flow through the flow sensor.

Nevertheless, other types of sensors could be used, for example pressure sensors.

In order to carry out the compliance evaluations, analysis means 40 are advantageously provided in order to analyse the measurements from each sensor 30 and thus to determine whether the impact zone of the flow of compressed gas F originating from said spray head 1 onto the reception surface 10 is or is not compliant with the predetermined specifications.

The duration of the compressed gas pulse F is advantageously adjustable, in particular from 50 to 300 ms.

Advantageously, a plurality of successive cycles are carried out on the same spray head, for example five cycles. The consistency or repeatability of the results also makes it possible to evaluate the compliance of said spray head.

The predetermined specifications may comprise a predetermined planar extent of said impact zone on said support surface 10, in a manner such that the spray heads 1 for which said planar extent is similar to said predetermined planar extent are classified as compliant, and the spray heads 1 for which said planar extent is different from said predetermined planar extent are classified as non-compliant. The geometry, and in particular the symmetry, of the impact zone may also be used in the compliance evaluation. Other parameters may also be envisaged, such as the differences between the measurements of adjacent sensors.

The means for analysis may comprise means for measuring the geometry of the impact zone of the flow of compressed gas F on the reception zone 10. As an example, the centre of mass of the impact zone is determined, and the maximum and minimum distances of this centre of mass from the edge of the impact zone are measured. Comparing these distances with predetermined values then makes it possible to evaluate the compliance of the tested device. Thus, the compliance evaluation takes not only the surface of the impact zone into account, but also its geometry, in particular its symmetry. This makes it possible to establish that a spray leaving a compliant spray head will have an acceptable conical shape, both from the point of view of the angle of the spray and as regards its symmetry.

FIG. 2 illustrates a schematic representation obtained with the method and the device of the invention, from which it is possible to evaluate in particular the planar extent and the geometry, in particular the symmetry, of the impact zone. Thus, in this example which shows a compliant result, it is found that the measurements of the different sensors are homogeneous in the spatial distribution, and with acceptable differences between the different concentric zones the array, which of demonstrates a good geometric distribution.

The present invention presents numerous advantages, and in particular:
it enables various types of spray device to be inspected in an automated manner;
it enables said spray devices to be analysed non-destructively;
it makes it possible to analyse 100% of the spray devices assembled on an assembly line, without slowing down the assembly line substantially;
it makes it possible to carry out several successive tests on the same device in order to evaluate the repeatability of the results;
it uses a setup that is compact and that can easily be adapted;
it uses components which are simple and standard, and thus generally inexpensive;
it enables image processing to be robust, and it can be carried out in real time;
it guarantees good repeatability and good discrimination between compliant and non-compliant devices.

The present invention has been described with reference to an advantageous embodiment, but naturally any modification could be applied thereto by the person skilled in the art, without going beyond the scope of the present invention, as defined by the accompanying claims.

1.-19. (canceled)

20. A method for analysing a fluid product spray device, the method comprising the following steps:
providing a spray head (1) for a device for spraying a pharmaceutical fluid product, said spray head (1) comprising a single spray orifice (2),
providing a receiving surface (10) comprising a plurality of sensors (20),
passing a flow of compressed gas (F) through said spray orifice (2) of said spray head (1),
sending said flow of compressed gas (F) onto said receiving surface (10),
visualising the impact zone for said flow of compressed gas (F) on said receiving surface (10) by means of said sensors (20), and
analysing said visualisation of said impact zone in order to determine whether or not said impact zone complies with predetermined specifications,
wherein said receiving surface (10) is at least partially spherical in shape, said sensors being arranged on the spherical part of said receiving surface, said step for analysis comprising determining the geometry, in particular the symmetry, of the impact zone for said flow of compressed gas (F) on said receiving surface (10).

21. The method according to claim 20, wherein said flow of compressed gas (F) is a flow of compressed air.

22. The method according to claim 20, wherein said predetermined specifications comprise a predetermined planar extent of the impact zone for said flow of compressed gas (F) onto said receiving surface (10), in a manner such that the spray heads (1) for which said planar extent is similar to said predetermined planar extent are classified as compliant, and the spray heads (1) for which said planar extent is different from said predetermined planar extent are classified as non-compliant.

23. The method according to claim 20, wherein said receiving surface (10) is formed by the inner surface of a part of a sphere, in particular a half-sphere.

24. The method according to claim 20, wherein said receiving surface (10) comprises a plurality of openings (11), each being connected to a respective sensor (20).

25. The method according to claim 20, wherein said sensors (20) form a honeycomb array comprising at least two concentric sensor zones.

26. A device for analysing a pharmaceutical fluid product spray device, comprising:
a spray head (1) for a device for spraying a pharmaceutical fluid product, said spray head (1) comprising a single spray orifice (2),
a receiving surface (10) comprising a plurality of sensors (20),
means (50) for generating a flow of compressed gas (F) in order to pass a flow of compressed gas (F) through said spray orifice (2) of said spray head (1) onto said receiving surface (10),
visualisation means in order to visualize through said sensors (20) the impact zone for said flow of compressed gas (F) onto said receiving surface (10), and
means (40) for analysis for the analysis of said visualisation of said impact zone in order to determine whether or not said impact zone complies with predetermined specifications,
characterised in that said receiving surface (10) is at least partially spherical in shape, said sensors being arranged on the spherical part of said receiving surface, said sensors (20) forming a honeycomb array comprising at least two concentric sensor zones.

27. The device according to claim 26, wherein said flow of compressed gas (F) is a flow of compressed air.

28. The device according to claim 27, wherein said receiving surface (10) is formed by the inner surface of a part of a sphere, in particular a half-sphere.

29. The device according to claim 27, wherein said receiving surface (10) comprises a plurality of openings (11), each being connected to a respective sensor (20).

30. The device according to claim 27, wherein a filter (30) is arranged in front of each sensor (20).

31. The device according to claim 26, wherein said sensor array (20) comprises a central area with a single central sensor, and a first concentric area, arranged radially outside of said central area, with six sensors.

32. The device according to claim 31, wherein said sensor array (20) comprises a second concentric zone, arranged radially outside of said first concentric zone, with twelve sensors.

33. The device according to claim 32, wherein said sensor array (20) comprises a third concentric zone, arranged radially outside of said second concentric zone, with eighteen sensors.

34. The device according to claim 26, wherein said sensors (20) are close to one another, thereby forming a regular and dense array of sensors (20) on said receiving surface (10).

35. The device according to claim 26, wherein said means (50) for generating a flow of compressed gas (F) are adapted to generate pulses of adjustable duration, in particular from 50 to 300 ms.

36. The device according to claim 26, wherein said sensors (20) are mass flow sensors.
